(19) United States
(12) Patent Application Publication
(10) Pub. No.: US 2014/0025062 A1
(43) Pub. Date: Jan. 23, 2014

(54) TREATMENT PROCESS INDICATOR

(71) Applicants: Avner Rosenberg, Bet Shearim (IL); Ulrich Toft, Moshav En Ayala (IL); Avi Magid, Givat Ela (IL)

(72) Inventors: Avner Rosenberg, Bet Shearim (IL); Ulrich Toft, Moshav En Ayala (IL); Avi Magid, Givat Ela (IL)

(73) Assignee: SYNERON MEDICAL LTD., Yoqneam Illit (IL)

(21) Appl. No.: 13/945,862

(22) Filed: Jul. 18, 2013

Related U.S. Application Data
(60) Provisional application No. 61/672,907, filed on Jul. 18, 2012.

(51) Int. Cl. A61B 18/18

(52) U.S. Cl. CPC A61B 18/18
USPC 606/33

(57) ABSTRACT

Described is an apparatus for fractional RF skin treatment that includes an RF generator and an applicator. The apparatus also includes a display facilitating display of RF skin treatment process results.

### CROSS-REFERENCE TO RELATED APPLICATIONS

This is a utility patent application being filed in the United States as a non-provisional application for patent under Title 35 U.S.C. §100 et seq. and 37 C.F.R. §1.53(b) and, claiming the benefit of the prior filing date under Title 35, U.S.C. §119(e) of the United States provisional application for patent that was filed on Jul. 18, 2012 and assigned Ser. No. 61/672,907, which application is incorporated herein by reference in its entirety.

### FIELD

The method and apparatus relate to treatment devices and in particular to skin treatment devices.

### BACKGROUND

External appearance is important practically to every person. In recent years, methods and devices have been developed for different cosmetic or aesthetic treatments Skin treatment by such device provides cosmetic procedures improving or maintaining person's external appearance. Typically, such products apply heat to different skin segments and heat the treated skin segment to a temperature causing the desired changes. Heat could be applied to the body by a source of Radio Frequency (RF), a source of UltraSound (US) energy, a source of optical energy or a combination of two or more sources listed above.

Most, if not all of these treatments are performed by professionals in dedicated cosmetic and body shaping clinics. Cosmetic body shaping treatments involving adipose tissue, also termed body contouring treatments, commonly involve employing complex devices and numerous methods of treatments to reduce body fat. These devices and treatments also include application of various forms of heating energy, mechanical energy and similar. The heating energy could be supplied in a continuous form or in pulse form.

The variety of skin types and skin conditions, and associated with them variations in skin treatment procedures, complicates almost every skin treatment as well as achievement of a desired skin effect or treatment result. Because of this, selection of proper skin treatment regimen is a trial and error process. The caregiver is basing the selection of skin treatment parameters on his experience. Typically, neither the caregiver nor the treated person receives instant feedback on the parameters selected and the success of the treatment. Naturally, it is clear that in a very few cases the skin treatment is performed with optimal skin treatment parameters.

### GLOSSARY

In the context of the present disclosure “RF voltage” and “RF energy” are used interchangeably and have the same meaning The mathematical relationship between these two parameters is well known and knowledge of one of them allows easy determination of the other.

In the context of the present disclosure “skin resistance” and “skin impedance” are used interchangeably and have the same meaning The mathematical relation between these two parameters is well known and knowledge of one of them allows easy determination of the other.

The term “desired skin effect” as used in the present disclosure means a result of RF energy application, which may be wrinkle treatment, hair removal/reduction, collagen shrinking or destruction, skin rejuvenation, and other cosmetic and skin treatments.

### SUMMARY

The present disclosure presents an apparatus and method for facilitating the proper selection of skin treatment parameters by a caregiver or by an individual that may be given self treatment. The parameters could include, as non-limiting examples, (a) the duration or amount of time that the skin heating energy is being applied, (b) the form in which the skin heating energy is supplied and/or (c) the type of the skin heating energy selected. The feedback to either or both the caregiver and the treated person could be provided in a variety of manners, such as but not limited to, at least one or both of visual, audio signals, tactile, etc. The visual signals could be in form of a graphic display by a series of color indicators visible to both the caregiver and the treated person, one or more LEDs or lights, or other illumination type devices as a non-limiting example.

According to one example, the apparatus may include at least a controller and an applicator. The controller communicates with the applicator and is configured to control operation of the applicator, including the supply of different types of skin treatment energy, power of the skin treatment energy, duration of the skin treatment energy application, and other parameters. The controller could operate to control different energy sources or generators, such as RF energy generators, ultrasound energy generators, mechanical energy generators, and optical energy generators. The energy generators could be located in a common housing with the controller or, the energy generators and controller can be housed in separate units. In some examples, both the controller and different energy sources could be located in a common housing, such as the applicator housing and/or with the applicator.

The controller could also include or communicate with a display to display in graphic or textual (alpha-numeric) form, the skin treatment process, the skin treatment energy parameters delivered to a treated skin segment and the results of the skin treatment process. To simplify the perception of the caregiver and the treated person, the display of the skin treatment results delivered to the treated skin segment skin treatment could be color coded, for example, the color green could be used to indicate optimal delivered to the treated skin segment skin treatment parameters, the color orange could be used to indicate that the skin treatment pulses delivered to the treated skin segment are not optimal (sub-optimal), and the color red could be used to indicate improper skin treatment parameters delivered to the treated skin segment.

A similar color coding system could be duplicated or ported to the applicator, for example, a green color LED could be used to indicate optimal delivered skin treatment energy parameters, an orange color LED could be used to indicate that the delivered skin treatment pulse parameters are not optimal, and a red color LED could be used to indicate an improper delivered skin treatment parameters. Thus the caregiver and the patient could easily monitor the treatment process without interrupting it and looking on the display. The mode of the LEDs operation, for example, blinking or continuously lighting an LED could be set to indicate additional treatment parameters, or in the alternative, the frequency and/or intensity of an illumination device can be used to convey all desired information. For instance, optimal delivery of the skin treatment parameters could be indicated by a gradual fade in and fade out of an LED while a sub-optimal delivery may be indicated by a steady on and improper delivery by a rapid flashing of the light. In some examples, a smaller size display could be located on the applicator.

According to some examples, the applicator could include tips for fractional skin treatment. The tips could include a first group or cluster of one or more large size voltage to skin delivering elements or electrodes located in the peripheral area of the tip and a second group of voltage to skin delivering elements or electrodes. The voltage to skin delivering elements of the second group includes discrete elements having an area smaller that the first elements have. The tips are configured to be applied to the skin.

According to some examples, the tips could have 64 smaller size elements, although other designs with different number of elements, for example 16, 40, 44, 64, 90 or 144 are possible. The tips could be of square, rectangular, oval or other geometrical shapes. The tips could be also termed as asymmetric tips, since the area of the first group of voltage to skin delivering elements is larger than the area of the second group of voltage to skin application elements or electrodes. The smaller or discrete electrodes could have a flat (pancake), needle or dome type shape.

### BRIEF LIST OF DRAWINGS

FIG. 1 is a schematic illustration of an apparatus for fractional skin treatment according to an example;

FIGS. 2A-2D are schematic illustrations of RF applicator tips for fractional skin treatment according to some examples; and

FIG. 3 is a simplified illustration of a display screen for ablative RF treatment screen according to an example.

### DESCRIPTION

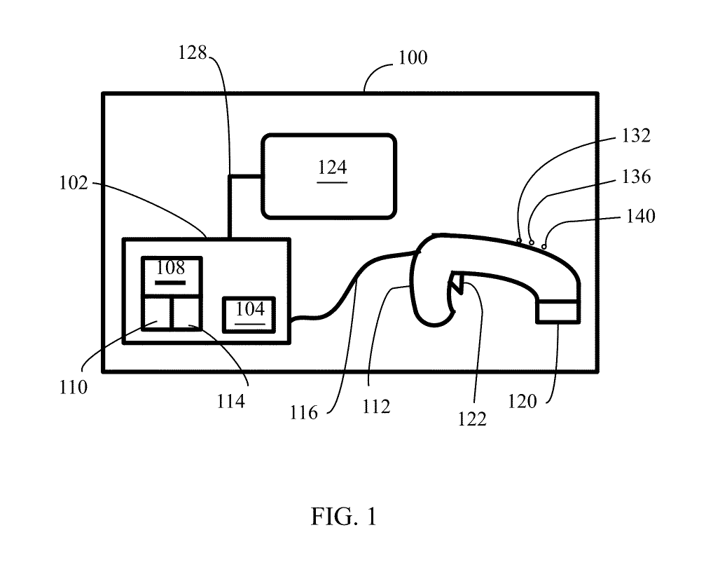

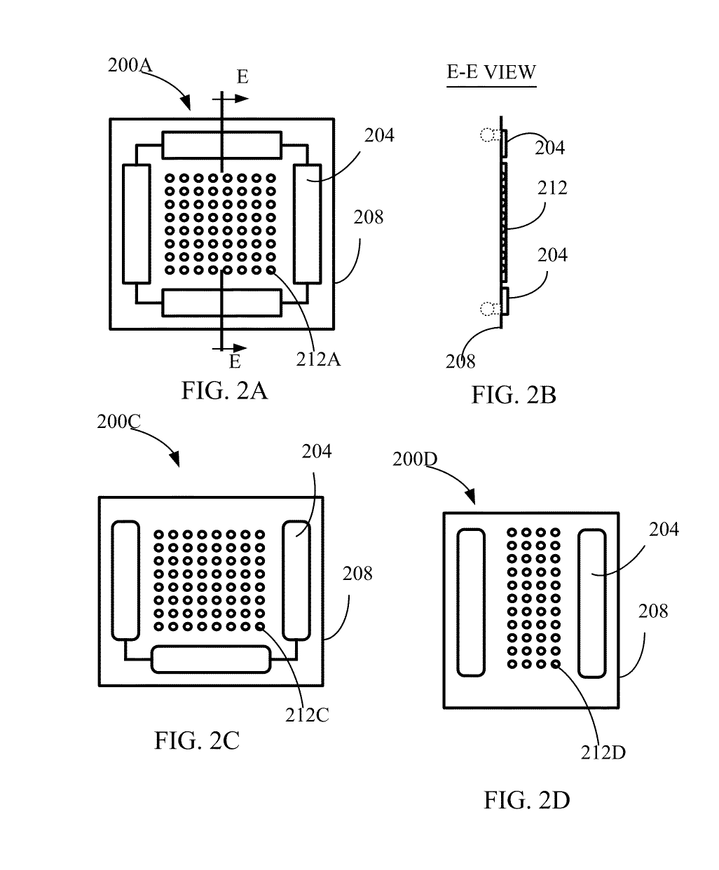

Reference is made to FIG. 1, which is a schematic illustration of an apparatus for fractional skin treatment according to an example. Apparatus 100 includes an RF voltage supply or generator 104, a controller 108, and an applicator 112. Both RF generator 104 and controller 108 may be located in the same housing 102, although they could be electrically and electromagnetically isolated to avoid electromagnetic interference between them. Controller 108 could include a processor 110 and a memory 114. In some examples controller 108 could be located in a common housing with an applicator 112. RF generator 104 could include a number of output ports. An umbilical cable 116 connects between applicator 112 and RF energy supply or generator 104. Applicator 112 is terminated by a tip 120 that, in the course of operation, is applied to a treated skin segment and delivers RF energy in pulse or continuous mode to that skin segment. Applicator tip 120 is a tip for fractional skin treatment and could be identical or similar to the tips shown in FIGS. 2, although other types of tips could be used. Trigger 122 could be used to activate RF energy supply to applicator tip 120. Umbilical cable 116 may conduct the voltage/energy supplied by the RF generator to the applicator. Cable 116 may be configured to include cooling fluid tubing and other tubes and/or wires that may be necessary to fulfill additional functions that could be of use in course of the treatment.

Apparatus 100 could further include a display 124. Display 124 communicates with controller 108 through cable 128. The display 124 could be a touch screen display operative to facilitate selection and setting of skin treatment parameters as well as to display the skin treatment process stages and the skin response to the treatment. Alternatively and optionally apparatus 100 could be operated through use of a keypad or keyboard.

Display of the skin treatment process stages provides a visual feedback with regards to the RF skin treatment results to the caregiver and to the treated person. The visual feedback could be in form of a color bar or strip, a color rectangle, a color circular shape or any other geometrical shape or form. A color coding system could be used to notify the caregiver and the patient of various status information. As a non-limiting example, different colors could represent the effectiveness of RF skin treatment pulse parameters delivered to a treated skin segment. As a more particular example, the color green, such as a green color strip, could be used to indicate that the RF skin treatment pulse parameters delivered to the treated skin segment are optimal; an orange color strip could be used to indicate that the RF skin treatment pulse parameters delivered to the treated skin segment are not optimal; and a red color strip could be used to indicate that the RF skin treatment pulse parameters delivered to the treated skin segment are improper. Controller 108 would interrupt or cut the “red” pulse of RF energy due to out-of-range skin tissue impedance. Because such pulse could result in causing damage to skin rather than a desired treatment result, the controller will automatically cut or discontinue such pulse/s. The caregiver and the treated person could easily monitor the skin treatment process by observing the display.

A similar color coding system could be duplicated or ported to the applicator 112. For example, lighting a green LED 132 could indicate an optimal delivery of skin treatment pulse parameters; an orange LED 136 could be used to indicate that the delivered skin treatment pulse parameters are not optimal; and a red LED 140 could be used to indicate an improper delivery of skin treatment pulse parameters. Thus, the caregiver and the patient could easily monitor the treatment process without interrupting it or even having to look on the display. The mode of the LEDs operation, for example, blinking or continuously lighting an LED could be set to indicate additional treatment parameters. In some examples, the three color LEDs 132, 136, and 140 could be replaced by a single LED capable of lighting in Green, Orange, and Red colors and other colors or indicators may also be used. For instance, alternatively and additionally audio indicators could be used in addition to the light indicators.

FIGS. 2A-2D are schematic illustrations of RF applicator tips for fractional skin treatment according to some examples. Each of the tips are illustrated as including a first group or cluster of larger “ground” voltage to skin delivering elements and a second group discrete elements that have or cover a smaller area. Tip 200A has a first group or cluster of four large “ground” voltage to skin delivering elements or electrodes 204 located in the peripheral area of a substrate 208 and a second group of voltage to skin delivering elements or electrodes 212. FIG. 2C illustrates a tip 200C that has a first group or cluster of three large ground voltage to skin delivering elements and FIG. 2D illustrates tip 200D that includes two large voltage to skin delivering elements. Each of the illustrated embodiments in FIG. 2A, 2B and 2C include the variations of a second group of electrodes shown as arrays of X by Y elements in array 212A, 212C and 212D (collectively or generically referred to as 212). The voltage to skin delivering elements 212 of the second group are discrete elements having an area smaller that the first element 204 has. The tip is configured to be applied to the skin. The configuration of the first and second group of electrodes can actually be presented on the substrate in any of the illustrated configurations as well as other configurations and the present disclosure is not limited to the illustrated embodiments. Rather, the shapes and relative spacing of the various elements can take on any of a variety of sizes and arrangements.

The first and the second group of electrodes could be connected to different ports of the output transformer of the RF generator 104. For example, the large “ground” electrodes could be connected to the first RF output port of RF generator 104 and the second group of smaller electrodes 212 could be connected to the other or second port of the RF output transformer. This particular output port of the transformer (not shown) may further be configured to have a plurality of output connections such as to support at least one different parameter of RF voltage supply to each of the smaller size discrete voltage to skin application elements or electrodes 212. A particular tip could have 64 elements, although other designs with different number of elements, for example 16, 40, 44, 64, or 144 are possible. The area of the first group of voltage to skin application elements 204 could be larger than the area of the second group of voltage to skin application elements or electrodes 212. The smaller or discrete electrodes may have a flat (pancake), needle or dome type shape, of diameter between 10 microns to 600 microns, or between 100 microns to 300 microns. The clusters of the electrodes and more specifically the smaller or discrete electrodes may be divided into sub-clusters, including sub-clusters with one electrode only, and each sub-cluster, including an individual electrode, may be driven by RF energy independent of the others and/or they can be operated sequentially, one after the other, or/and they can be operated concurrently.

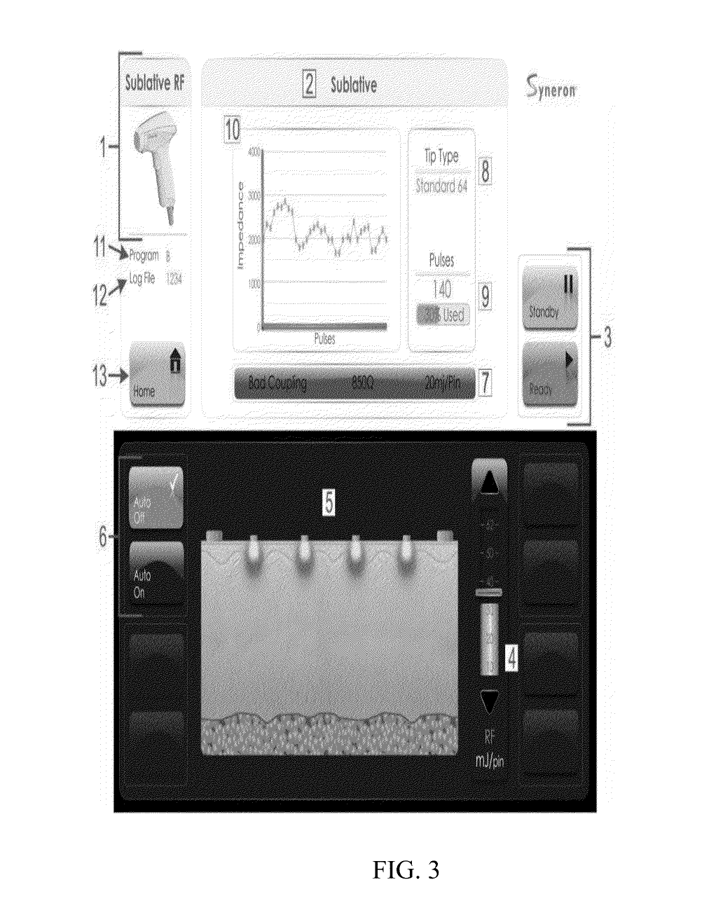

FIG. 3 is a simplified illustration of a display screen for ablative RF treatment according to an example. The numbered tags in FIG. 3 correspond to the following definitions of the displayed on the screens elements that are available in the illustrated embodiment but, that do not limit or identified required display screen elements:

Number 1 indicates and illustrates the currently active applicator.

Number 2 describes the function of the currently active applicator.

Number 3 relates to Standby and Ready Buttons. A screen touch of the Ready Button sets the apparatus to Ready mode; RF energy could be emitted and coupled to skin through the applicator tip 120 when the trigger 122 (FIG. 1) is pressed. Touching the screen at the image of the Standby button will set the apparatus back to Standby mode. When the apparatus is in Ready mode, pressing any other button will automatically set the system back to Standby mode.

Number 4 is the RF Energy Selection Bar: pressing anywhere inside the bar, or on the ▴ or ▾ buttons will increase or decrease the RF energy (mJ/pin) supplied to the discrete electrodes. The energy values could be within the Syneron recommended range of RF energy skin treatment parameters indicated by a green area inside the RF selection bar. Alternatively and optionally the caregiver can use a wide range of different than recommended RF energy values.

Number 5 illustrates the effect that RF energy applied to the skin could perform. The effect is displayed as the relative depth of the RF energy penetration into the skin. The display changes as the level of applied RF energy is adjusted automatically by the controller or by the caregiver.

Number 6 indicates the display screen segment dedicated to Auto OFF and Auto ON Buttons. When active (Auto ON), the apparatus will emit pulses continuously for as long as the trigger is pressed at a rate of 1 Hz (1 pulse per second). When inactive (Auto OFF), the trigger must be manually pressed for each pulse.

Number 7 is a Skin Feedback Bar. Following RF pulse application, this bar exhibits pulse measured indications: an alert message (when applicable), the skin's impedance (measured in Ohms) and the delivered RF energy. The background color of the bar may alternate between Green, Orange or Red.

Number 8 indicates the tip currently inserted into the applicator and used for the skin treatment.

Number 9 is the applicator tip Pulse Life Indicator: each new treatment tip is valid for up-to 400 pulses (tip type dependent). When a new tip is connected the bar starts at 0%, and progresses up as the tip is used; for example: a 50% Used message means that 100 of the 200-pulse tip's pulses have been emitted.

Number 10 is an illustration of the Skin Impedance/Pulses Graph. The graph displays a forward-moving average of the impedance measured by the pulses. Each point in the graph on the Y-axis represents the average impedance of the previous five pulses; the next point of the graph will show the average impedance of the previous five pulses.

During the first four pulses of the treatment session—before the fifth average can be established each point on the graph represents the measured impedance of only that pulse. The moving average begins at the fifth pulse emitted. The measured impedance of each of the pulses is displayed on the skin feedback bar (Number 7) in Ohms.

For RF skin treatment, applicator 112 (FIG. 1) is applied to a segment of skin to be treated. The caregiver could use display 124 and in particular the RF Energy Selection Bar (Number 4 in FIG. 3) to set the desired RF skin treatment parameters and in particular the RF energy (mJ/pin). The energy values could be within the Syneron Medical Ltd. (Syneron), recommended range of RF energy indicated by a green area inside the RF energy selection bar. The Syneron recommended RF skin treatment energy parameters could be prepared by the apparatus 100 operator or caregiver ahead of time and loaded into a look-up-table (LUT) that could be stored in the memory 114 of the controller 108 (FIG. 1).

Following RF pulse application, display 124 displays the color coded Skin Feedback Bar (Number 7, FIG. 3) as a rectangular strip or bar, although it should be understood that other color coded shapes such as a square, a circle, regular or even irregular geometrical shapes could be used to the display the RF skin treatment process results.

According to an example, the shapes could be color shapes and their color could consist of a green color, orange color or red color. The green color could indicate the optimal delivery of RF skin treatment pulse parameters to the treated skin segment matching the impedance of the treated skin segment. The orange color could indicate that the RF skin treatment pulse parameters delivered to the treated skin segment are not optimal (sub-optimal). The red color could indicate that improper RF skin treatment pulse parameters have been delivered to the treated skin segment. Both the caregiver and the treated person could observe the display 124 and follow the treatment progress. Following first skin treatment, RF energy application and results of the treatment are displayed in color, the caregiver or operator could act to change the RF energy to optimal RF skin treatment settings—green Skin Feedback Bar. The decision on optimal, sub-optimal, and improper includes RF treatment energy considerations and skin resistance (or impedance) which was found to be related to the physical processes at the skin.

The displayed shape/s could include an alpha-numeric message or a group of alpha-numeric messages. The message could be an alert message. The message could include skin impedance indication and delivered to the skin RF energy.

In cases of skin treatment where observation of the display 124 is not convenient, the caregiver and the treated person could observe the applicator 112 that includes three colors LEDs 132, 136, and 140 (or one color LED lighting by three colors) lighting with identical to the display colors. The controller synchronizes operation of the display of the shapes and their color and the operation and color of the LEDs on the applicator. In some examples, a small size display could be attached to the applicator to display at least part of the information on the main screen or to replace the main screen.

Number 9 is a tip Pulse Life Indicator: each new treatment tip is valid for up-to 400 pulses (tip type dependent). When a new tip is connected the bar starts at 0%, and progresses up as the tip is used; for example: a 50% Used message means that 100 of the 200-pulse tip's pulses have been emitted. Both the caregiver and the treated person could track applicator state.

What is claimed is:

1. An apparatus for fractional RF skin treatment, said apparatus comprising:
an RF generator and an applicator including a tip, wherein the tip comprises at least one first voltage to skin delivering element and at least one second voltage to skin delivering element, each second voltage to skin delivering element having an area smaller than each of first voltage to skin delivering element, the tip configured to be applied to the skin;
an interface enabling at least the function of setting of RF skin treatment parameters; and
a controller operative to receive the RF skin treatment parameters, monitor the treatment process and communicate RF skin treatment process results to the interface; and
wherein the interface includes a display element and the RF skin treatment process results are displayed in a numeric or graphic form.

2. The apparatus according to claim 1, wherein the display includes indicators to show if the RF skin treatment process results from an optimal RF skin treatment energy pulse, a non-optimal RF skin treatment energy pulse, and an improper RF skin treatment energy pulse.

3. The apparatus according to claim 1, wherein the display includes indicators in at least one of a group of shapes consisting of a rectangular strip/bar, a square, a circle or any other geometrical shape including irregular shape for the display of the RF skin treatment process results.

4. The apparatus according to claim 3, wherein the shapes include at least one of a group of alpha-numeric messages consisting of an alert message, skin impedance and RF energy delivered to treated skin segment.

5. The apparatus according to claim 3, wherein the shapes are of at least one of group of consisting of a green, orange and red.

6. The apparatus according to claim 5, wherein the green color indicates optimal delivery of RF skin treatment energy pulse.

7. The apparatus according to claim 5, wherein the orange color indicates that delivered RF skin treatment energy pulse was not an optimal pulse.

8. The apparatus according to claim 5, wherein the red color indicates an interrupted or cut RF skin treatment energy pulse due to out-of-range skin impedance.

9. The apparatus according to claim 1, wherein the RF skin treatment parameters include at least RF energy (mJ/pin) supplied to the second voltage to skin delivering element.

10. The apparatus according to claim 1, wherein recommended RF energy values are selected from a green color-coded area of recommended RF energy values or a wider range of different RF energy values.

11. The apparatus according to claim 1, wherein the RF skin treatment process results are displayed as a relative depth of RF skin treatment energy penetration into the skin.

12. The apparatus according to claim 1, wherein the applicator includes at least three LEDs and wherein at least one of the three LEDs lights in green color, at least one of the three LEDs lights in orange color, and at least one of the three LEDs lights in red color.

13. The apparatus according to claim 12, wherein in operation, the color of the shapes on the display and the color of the LEDs on the applicator are synchronized.

14. An applicator for fractional RF skin treatment, said applicator comprising:
a tip with at least one first voltage to skin delivering element and one second voltage to skin delivering element having an area smaller that the first voltage to skin delivering element, the tip configured to be applied to the skin;
a display device that includes at least three states, wherein the RF skin treatment process results are displayed by causing the display device to transition to a state that represents the RF skin treat process results.

15. The applicator according to claim 14, wherein the display device includes at least three LEDs and wherein at least one of the three LEDs lights in green color, at least one of the three LEDs lights in orange color, and at least one of the three LEDs lights in red color; and illuminating the green colored LED indicates a state that represents optimal delivery of RF energy

16. The applicator according to claim 14, wherein the display device includes at least three LEDs and wherein at least one of the three LEDs lights in green color, at least one of the three LEDs lights in orange color, and at least one of the three LEDs lights in red color; and illuminating the orange color LED indicates a state that represents that emitted RF skin treatment pulse was not optimal pulse.

17. The applicator according to claim 14, wherein the display device includes at least three LEDs and wherein at least one of the three LEDs lights in green color, at least one of the three LEDs lights in orange color, and at least one of the three LEDs lights in red color; and illuminating the red colored LED indicates a state that represents that an interrupted or cut pulse of RF energy due to out-of-range skin impedance.

18. The applicator according to claim 14, wherein the display device includes one LED configured to illuminate in at least three colors such as green, orange, and red replaces the three color LEDs.

19. A method for fractional RF skin treatment, said method comprising:
applying to a segment of skin to be treated an applicator including a tip with at least one first voltage to skin delivering element and one second voltage to skin delivering element having an area smaller that the first one;
supplying RF energy to the at least one voltage to skin delivering element and at least one second voltage to skin delivering element; and
displaying in a numeric or graphic form at least RF skin treatment results.
